(19) United States
(12) Patent Application Publication
(10) Pub. No.: US 2003/0211136 A1
(43) Pub. Date: Nov. 13, 2003

(54) Fast dissolving orally consumable films containing a sweetener

(76) Inventors: Neema Kulkarni, Randolph, NJ (US); Lori D. Kumar, Skillman, NJ (US); Albert Sorg, Columbia, NJ (US)

Correspondence Address: Barry H. Jacobsen, Warner-Lambert Company LLC, 201 Tabor Rd., 56-2 South, Morris Plains, NJ, 07960, US

(21) Appl. No.: 10/423,398

(22) Filed: Apr. 25, 2003

Related U.S. Application Data
(63) Continuation-in-part of application No. 09/395,104, filed on Sep. 14, 1999, now Pat. No. 6,596,298.
(60) Provisional application No. 60/101,798, filed on Sep. 25, 1998.

(51) Int. Cl. A61K 31/7012; A61K 31/485; A61K 31/44; A61K 31/60; A61K 31/415; A61K 31/405

(52) U.S. Cl. 424/443; 514/53; 514/165; 514/569; 514/570; 514/282; 514/649; 514/420; 514/406; 514/358

(57) ABSTRACT

A consumable film adapted to adhere to and dissolve in the oral cavity of a warm-blooded animal including humans, comprising at least one water soluble polymer, a taste masking effective amount of a sweetener, and a pharmaceutically active agent having a sufficiently unpleasant taste that it is desirably masked by the sweetener.

### FIELD OF THE INVENTION

The present invention is related generally to fast dissolving orally consumable films for delivering one or more pharmaceutically active agents, and more particularly to fast dissolving orally consumable films containing a sweetener for improving the taste of the film.

### BACKGROUND OF RELATED TECHNOLOGIES

Personal care products can be formulated in a variety of dosage forms, including tablets, capsules, lozenges or strips of edible thin film compositions. Edible thin film compositions applied to the oral cavity can be designed to deliver therapeutic agents to the oral mucosa. One such example is LISTERINE POCKETPAKS™ brand oral care strip products made by Pfizer Inc. of New York are successful examples of an edible film compositions effective in delivering therapeutic agents particularly antimicrobial agents in the form of a combination of essential oils.

Conventional rapidly dissolving orally consumable films may have incorporated flavorants and/or sweetening agents to improve the taste of the film and/or its components (e.g., pharmaceutically active agents) contained therein. The flavorants and/or sweetening agents used in such films generally provide limited taste improvement especially for films containing bitter tasting components. Accordingly, there still remains a need in the art to develop consumable thin films containing a sweetener, which at least substantially improves the taste of films and its components.

### SUMMARY

One embodiment of the present invention provides a consumable film adapted to adhere to and dissolve in the oral cavity of a warm-blooded animal including humans, which comprises at least one water soluble polymer, a taste masking effective amount of a sweetener, and a pharmaceutically active agent having a sufficiently unpleasant taste that it is desirably masked by the sweetener. In another embodiment of the present invention, there is provided a consumable film adapted to adhere to and dissolve in the oral cavity of a warm-blooded animal including humans, comprising at least one water soluble polymer, a taste masking effective amount of a sucralose, and a pharmaceutically active agent.

The present invention is also directed to a method of preparing a supple, non-self-adhering film especially suitable for oral delivery of pharmaceutically active agents where the method comprises preparing a film-forming mixture including at least one water soluble polymer; preparing an aqueous phase comprising a sweetener and a pharmaceutically active agent; combining the aqueous phase and the film forming mixture to form a hydrated polymer gel; casting the hydrated polymer gel on a substrate to form a cast gel; and drying the cast gel to form the consumable film.

### DETAILED DESCRIPTION OF THE INVENTION

An embodiment of the present invention is directed to a physiologically acceptable film that is well-adapted to dissolve in the oral cavity of a warm-blooded animal including humans afflicted with a disease, symptom or condition, and adhere to the mucosa of the oral cavity. Such films are suited to deliver a pharmaceutically active agent useful for treating the afflicted warm-blooded animal.

In one aspect of the present invention, there is provided a consumable film adapted to adhere to and dissolve in the mouth of a warm-blooded animal including humans, comprising at least one water soluble polymer, a taste masking effective amount of a sweetener, and a pharmaceutically active agent having a sufficiently unpleasant taste that it is desirably masked by the sweetener.

The consumable film may include one or more of the following ingredients, including, but not limited to, water, antimicrobial agents, additional film forming agents or water soluble polymers, plasticizing agents, flavorings, sulfur precipitating agents, saliva stimulating agents, cooling agents, surfactants, stabilizing agents, emulsifying agents, thickening agents, binding agents, coloring agents, triglycerides, polyethylene oxides, propylene glycols, sweeteners, fragrances, preservatives and the like, as described in co-pending application U.S. patent application Ser. No. 09/395,104, by Leung et al., filed Sep. 14, 1999, which is incorporated herein by reference in its entirety.

In one embodiment of the present invention, the consumable film is in the form of a single layer.

The term “consumable” as used herein is intended to encompass substances including edible compounds, which upon administration to a consumer, is adequately tolerated without causing undue negative effects. Consumable films are shaped and sized for administration to the oral cavity of a warm-blooded animal including humans. The films are particularly well adapted to rapidly dissolve in the mouth of the warm-blooded animal. The dissolved film adheres to the surface of the mouth, typically the roof of the mouth or the tongue, and can provide a rapid delivery system for pharmaceutically active agents.

Unless specified otherwise, the term “% by weight” as used herein with reference to the final product (i.e., the film, as opposed to the formulation used to produce the film), denotes the percent of the total dry weight contributed by the subject ingredient. This theoretical value can differ from the experimental value, because in practice, the film typically retains some of the water and/or other substances such as alcohols (e.g., ethanol) that may be used in preparing the final product.

In one embodiment, the consumable film of the present invention includes a pharmaceutically active agent and a sweetener that significantly improves the taste of the pharmaceutically active agent for enhanced product performance and consumer acceptance. By improving the taste of the films containing pharmaceutically active agents in accordance with the present invention, compliance and adherence to treatments involving such films would be significantly enhanced. Suitable sweeteners include natural and artificial sweeteners.

Useful sweetening agents include A) water-soluble sweetening agents such as, for example, monosaccharides, disaccharides and polysaccharides, B) water-soluble artificial sweetening agents such as, for example, soluble saccharin salts and the like, C) dipeptide based sweetening agents such as L-aspartic acid derived sweetening agents and the like, D) protein based sweeteners such as, for example, thaumatoccous danielli (Thaumatin I and II), and mixtures thereof. Additional suitable sweeteners include sucralose, aspartame, acesulfame potassium, neotame, saccharin, xylitol and mixtures thereof.

The sweetener is employed in an effective amount, which will vary depending in part on the specific sweetener chosen. A “taste masking effective amount” is meant to be an amount of the sweetener that is sufficient to at least reduce, mask or eliminate the unpleasant taste (e.g., bitter) of the pharmaceutically active agent contained in the film of the present invention. In addition to the particular sweetener, the taste masking effective amount may vary with the type and/or the degree of the taste being masked and the particular carrier and ingredients contained in the film. In one embodiment, the sweetener may be present in the dry film of the present invention in taste masking effective amounts ranging from about 0.1% to 10% by weight, preferably 1% to 6% by weight, and more preferably from about 2% to 4% by weight of the film.

One embodiment includes sucralose as a sweetener. Sucralose is a chlorinated sucrose derivative having an intensely sweet taste. Sucralose has been discovered to effectively mask or nullify the unpleasant taste attributes of many food additives and pharmaceutically active agents especially those that are bitter tasting. By incorporating sucralose into the films of the present invention, enhanced sweetness and desirable masking of any unpleasant taste supplanted by food additives and pharmaceutically active agents (e.g., dextromethorphan hydrobromide, famotidine) that may be contained therein, will be beneficially realized.

The water soluble polymers of the present invention possess film forming properties useful producing the films of the present invention. The water soluble polymer used in the films of the present invention can be selected from the group consisting of pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymers, carboxyvinyl polymers, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. In one embodiment of the present invention the water soluble polymer is pullulan which may be present in amounts ranging from about 0.01% to 99% by weight, in another embodiment from about 10% to 80% by weight, in another embodiment from about 20% to 70% by weight of the film and in yet another embodiment from about 30% to 50% by weight of the film.

The term “pharmaceutically active agents” as used herein is intended to encompass agents other than food additives, which promote a structural and/or functional change in and/or on bodies to which they have been administered. These agents are not particularly limited, however, they should be physiologically acceptable and compatible with the film. Suitable pharmaceutically active agents that may be unpleasant to the taste, include, but are not limited to,

(a) antimicrobial agents such as triclosan, cetyl pyridium chloride, domiphen bromide, quaternary ammonium salts, zinc compounds, sanguinarine, fluorides, alexidine, octonidine, EDTA, and the like;

(b) non-steroidal anti-inflammatory drugs such as aspirin, acetaminophen, ibuprofen, ketoprofen, diflunisal, fenoprofen calcium, flurbiprofen sodium, naproxen, tolmetin sodium, indomethacin, celecoxib, rofecoxib and the like;

(c) antitussives such as benzonatate, caramiphen edisylate, menthol, dextromethorphan hydrobromide, chlophedianol hydrochloride and the like;

(d) decongestants such as pseudoephedrine hydrochloride, phenylepherine, phenylpropanolamine, pseudoephedrine sulfate and the like;

(e) antihistamines such as brompheniramine maleate, chlorpheniramine maleate, carbinoxamine maleate, clemastine fumarate, dexchlorpheniramine maleate, diphenylhydramine hydrochloride, azatadine maleate, diphenhydramine citrate, diphenylpyraline hydrochloride, doxylamine succinate, promethazine hydrochloride, pyrilamine maleate, tripelennamine citrate, triprolidine hydrochloride, acrivastine, brompheniramine, dexbropheniramine, fexofenadine, loratadine, cetirizine, and the like;

(f) expectorants such as guaifenesin, ipecac, potassium iodide, terpin hydrate and the like;

(g) antidiarrheals such as loperamide and the like;

(h) histamine II receptor antagonists such as famotidine, ranitidine and the like;

(i) proton pump inhibitors such as omerprazole, lansoprazole and the like;

(j) general nonselective CNS depressants such as aliphatic alcohols, barbiturates and the like;

(k) general nonselective CNS stimulants such as caffeine, nicotine, strychnine, picrotoxin, pentylenetetrazol and the like;

(l) drugs that selectively modify CNS function such as phenyhydantoin, phenobarbital, primidone, carbamazepine, ethosuximide, methsuximide, phensuximide, trimethadione, diazepam, benzodiazepines, phenacemide, pheneturide, acetazolamide, sulthiame bromide, gabapentin, phenytoin and the like;

(m) antiparkinsonism drugs such as levodopa, amantadine and the like;

(n) narcotic-analgesics such as morphine, heroin, hydromorphone, metopon, oxymorphone, levorphanol, codeine, hydrocodone, oxycodone, nalorphine, naloxone, naltrexone and the like;

(o) analgesic-antipyretics such salicylates, phenylbutazone, indomethacin, phenacetin and the like; and

(p) psychopharmacological drugs such as chlorpromazine, methotrimeprazine, haloperidol, clozapine, reserpine, imipramine, tranylcypromine, pheneizine, lithium and the like.

The pharmaceutically active agent is employed in an effective amount, which will vary depending, in part on the pharmaceutically active agent chosen. An “effective amount” is meant to be an amount of the pharmaceutically active agent that sufficient to at least reduce or relieve the condition, symptom or disease being treated, but low enough to avoid any adverse side effects. In addition to the particular active agent, the effective amount of the pharmaceutically active agent may vary with the type and/or severity of the disease, symptom or condition, the age and physical condition of the patient being treated, the duration of treatment, the nature of concurrent therapy, the specific form (i.e., salt) of the pharmaceutically active agent employed, and the particular carrier from which the pharmaceutically active agent is applied.

The amount of the pharmaceutically active agent in the formulation may be adjusted to deliver a predetermined dose of the pharmaceutically active agent over a predetermined period of time, which may typically vary from 4 to 24 hours. For example, in one embodiment of the present invention, the film may be administered at one dose every 12 hours to deliver a pharmaceutically effective amount of the pharmaceutically active agent such as dextromethorphan, for example, over a period of 12 hours to a patient in need of such administration. A typical adult dose of a pharmaceutically active agent of the present film may contain from about 0.1 to 130 mg, preferably from about 0.1 to 65 mg of the pharmaceutically active agent (e.g., dextromethorphan hydrobromide).

Examples of doses for specific pharmaceutically active agents that can be delivered per one strip of rapidly dissolving oral film are reviewed in Table A.

TABLE A

| Pharmaceutically Active Agent | Dose | |
|---|---|---|
| Chlorpheniramine Maleate | 4-12 | mg |
| Brompheniramine Maleate | 4 | mg |
| Dexchlorpheniramine | 2 | mg |
| Dexbropheniramine | 2 | mg |
| Triprolidine Hydrochloride | 2.5 | mg |
| Cetirizine | 5-10 | mg |
| Acrivastine | 8 | mg |
| Azatadine Maleate | 1 | mg |
| Loratadine | 5-10 | mg |
| Phenylephrine Hydrochloride | 5-10 | mg |
| Dextromethorphan Hydrobromide | 10-30 | mg |
| Sildenafil | 25-100 | mg |
| Ketoprofen | 12.5-25 | mg |
| Sumatriptan Succinate | 35-70 | mg |
| Zolmitriptan | 2.5 | mg |
| Loperamide | 2 | mg |
| Famotidine | 5-10 | mg |
| Nicotine | 1-15 | mg |
| Diphenhydramine Hydrochloride | 12.5-25 | mg |
| Pseudoephedrine Hydrochloride | 15-60 | mg |
| Atorvastatin | 5-80 | mg |
| Valdecoxib | 5-20 | mg |
| Amlodipine besylate | 2.5-10 | mg |
| Rofecoxib | 5-25 | mg |
| Setraline hydrochloride | 10-100 | mg |
| Ziprasidone | 20-80 | mg |
| Eletriptan | 10-40 | mg |
| Nitroglycerin | 0.3-0.6 | mg |

Except as otherwise noted, the amount of active agent in the film according to the present invention is designated as % by weight after the film formulation has been dried and formed into the film. Generally, the amount of the active agent used in the film may be from about 0.01% to about 80% by weight, preferably from about 2.5% to about 40% by weight, and more preferably from about 5% to about 30% by weight.

The film compositions of the present invention may also be used to supply nutritionally acceptable components such as vitamins, minerals, trace elements, and fibers (preferably soluble fibers).

Examples of vitamins suitable for the incorporation in the composition of the invention include Vitamin A, Vitamin D, Vitamin E, Vitamin K, Vitamin C, folic acid, 10 thiamin, riboflavin, Vitamin B (6), Vitamin B (12), niacin, biotin and panthotenic acid in pharmaceutical or nutritionally acceptable form. Examples of mineral elements and trace elements suitable for the incorporation in the composition of the invention include calcium, sodium, potassium, phosphorous, magnesium, manganese, copper, zinc, iron, selenium, chromium and molybdenum in pharmaceutical or nutritionally acceptable form.

The term soluble fiber as used herein refers to fibers which are able to substantially undergo fermentation in the colon to produce short chain fatty acids.

Examples of suitable soluble fibers include, carubin, pectin, tragacanth, cereal beta-glucan and the like. They may be hydrolysed or not.

In another embodiment of the present invention, the consumable film may further include antimicrobial agents including, but not limited to, essential oils as is described in co-pending U.S. patent application Ser. No. 09/395,104, by Leung et al., filed Sep. 14, 1999, which is incorporated herein by reference in its entirety. Such essential oils may be selected from, for example, carvacrol, thymol, eucalyptol, menthol, methyl salicylate, eugenol, gerianol, verbenone and the like and combinations thereof. One of the preferred combinations of essential oils is utilized in LISTERINE® brand mouthwash and oral care strips, which are, perhaps, the most well known examples of antiseptic oral compositions that has proven effective in killing microorganisms in the oral cavity that are responsible for plaque, gingivitis and bad breath. LISTERINE® brand mouthwash and oral care strips achieve their antimicrobial effect through a combination of essential oils. These essential oils include precisely balanced amounts of thymol, methyl salicylate, menthol and eucalyptol (hereinafter “the preferred essential oils”) effective in killing the undesirable microorganisms.

The amounts of the preferred essential oils used in the film compositions can vary as long as they are in amounts sufficient to provide antimicrobial efficacy. Generally, the amount of essential oils is up to about 30% and preferably from about 0.05% to about 18% by weight of the film. In one preferred embodiment, the amount of thymol, methyl salicylate and eucalyptol is each from about 0.01% to about 4% by weight, preferably from about 0.05% to about 3.0% by weight and more preferably from about 0.07% to about 2.0% by weight of the film. Menthol may be present in an amount of from about 0.01% to about 15% by weight of the composition, preferably from about 2.0% to about 9.0% by weight and more preferably from about 3% to about 9% by weight of the film. A desirable and useful amount of essential oils including the preferred essential oils can be readily determined by those skilled in the art and may exceed the preferred amounts as long as the total essential oil content does not create processing problems such as sticking. In certain embodiments, the essential oils are combined in amounts synergistically effective to kill plaque-producing germs that cause dental plaque, gingivitis and bad breath.

For embodiments incorporating essential oils, humectants are avoided due to the relatively high content of oil in the consumable, so as to avoid producing an overly moist, self-adhering film. In an embodiment, the consumable film includes a plasticizing agent other than glycerin, which is also a humectant, and with a sweetener other than sorbitol, which is a mild humectant.

Saliva stimulating agents may also be added to the consumable films of the present invention. Useful saliva stimulating agents are disclosed in U.S. Pat. No. 4,820,506, which is incorporated herein by reference in its entirety.

The consumable films of the present invention may also include a preservative. The preservative is added in amounts up to about 5%, preferably from about 0.01% to 1% by weight of the film. Preferred preservatives include sodium benzoate, methyl parabens, propyl parabens and potassium sorbate. Other suitable preservatives include, but are not limited to, salts of edetate, (also known as salts of ethylenediaminetetraacetic acid, or EDTA, such a disodium EDTA).

Another embodiment of the present invention is directed to methods of preparing consumable films of the present invention. Generally, at least one antitussive agent and a mucosa-coating effective amount of a mucosa-coating agent are dissolved in water to form an aqueous phase. The aqueous phase may further include sweeteners, dyes, and the like. A film forming mixture comprising at least one water soluble polymer (e.g., pullulan) is prepared. The aqueous phase and the film forming mixture are combined and thoroughly mixed to form a hydrated polymer gel. Optionally, an organic phase comprising organic ingredients such as essential oils and other oils (e.g. glycerine, olive oil) flavorants, surfactants (e.g., Polysorbate 80, Atmos 300, Atsurf 596K); and the like, may be combined with the aqueous phase, the film forming mixture or the hydrated polymer gel. The resulting hydrated polymer gel is cast on a suitable substrate to form a cast gel. The cast gel is then dried to form the consumable film.

In another embodiment there is provided a method of preparing the consumable film, it may be desirable to first form the film forming mixture by first hydrating the water soluble polymer with water. The aqueous phase is then prepared by dissolving the other water soluble ingredients such as the antitussive agent, the mucosa-coating agent (e.g., pectin), sweeteners, dyes, and the like in water. Separately, the organic ingredients such as essential oils and other oils (e.g. glycerine, olive oil) flavorants, surfactants (e.g., Polysorbate 80, Atmos 300, Atsurf 596K); and the like are mixed together. The final formulation is then produced by mixing the film forming polymer phase with the aqueous phase, then adding the organic phase. The combined mixture is formed into an emulsion or a hydrated polymer gel.

The resulting hydrated polymer gel is then cast on a suitable substrate and dried to form a film. The consumable film is preferably air-dried and dried under warm air and cut to a desired dimension, packaged and stored. The packaged film may contain moisture in amounts of from about 0.1% to about 10% by weight, and more preferably from about 4% to about 7% by weight.

The film forming mixture may further include stabilizing agents such as xanthan gum, locust bean gum, carrageenan, and the like, and combinations thereof. These ingredients are mixed and then hydrated in warm water, preferably deionized water until a gel is formed which may take from about 30 to about 48 hours. The water is preferably heated to a temperature of from about 20° C. to about 40° C. to promote hydration. The amount of water is typically from about 40% to about 80% by weight of the gel. The resulting hydrated gel is then chilled to a temperature of from about 20° C. to about 30° C. for about 1 hour to about 48 hours.

The aqueous phase may, in addition to the antitussive agent and the mucosa coating effective amount of the mucosa-coating agent such as pectin, include additives such as coloring agents, copper gluconate and sweetener. Typically the aqueous phase contains from about 5% to about 80% by weight based on the total weight of the final gel mixture.

If sodium saccharin as a selected sweetener and copper gluconate as a selected sulfur precipitating agent are used in the formulation, it is preferable to dissolve them separately in solution to avoid precipitation.

In another embodiment of the present invention, the water soluble polymer is in the form of a powder which is added to the aqueous phase to form a hydrated polymer gel. The resulting hydrated polymer gel is thoroughly stirred at about room temperature for about 30 minutes to about 48 hours, and then deaerated to remove at least substantially all the air bubbles. The uniform mixture is cast on a suitable substrate, and thereafter dried to form the desired film.

For consumable films containing essential oils, the essential oils are further added to the organic phase and the mixing the organic phase with the hydrated polymer gel. In particular, the essential oils such as menthol and thymol can be mixed optionally in combination with oils to form an oil mixture. Other essentials oils such as methyl salicylate and eucalyptol, and surfactants can then be added to the oil mixture. The oil mixture is then added to the hydrated polymer gel and mixed until a uniform gel is formed. The uniform gel is then cast on a suitable substrate, and thereafter dried to form the consumable film.

In one embodiment for preparing the consumable film, the water soluble polymer may be hydrated without heating the water to reduce energy costs in the manufacturing process. Moreover, since heating may result in undesirable losses of volatile ingredients to evaporation, it would be preferable to avoid heating during the hydration process. For essential oil-containing films, the heat may also affect the germ killing activity of the composition due to the loss of essential oils.

While not wishing to be bound by any theory, it is believed that the film forming ingredients such as the water soluble polymers can be hydrated and mixed without heating due to an ionic effect known as the Donnan equilibrium. Hydrating the water soluble polymers in the presence of electrolytes in solution effectively lowers the viscosity of the polymer gel being formed, thus increasing the efficiency of the hydrating process. The water-soluble ingredients of the formulation provide the electrolytes, which are dissolved in the hydration solution prior to addition to the water-soluble polymers. High shear mixing also accelerates hydration, which delumps the powders, providing greater surface area for water contact. In addition, local heating effects, generated in the shear regions, provide energy for hydration without substantially raising the temperature of the mass.

### EXAMPLE 1

The ingredients listed in Table 1 were combined to provide a consumable film of the present invention in accordance with the following procedure:

A) Dextromethorphan HBr was mixed and dissolved in 90% water at 75° C. to yield an aqueous phase. Amberlite IRP69 was added to the aqueous phase and stirred for about 4 to 5 hours at about 70° C. to 8020 C. The aqueous phase was allowed to cool to about 50° C. and q.s. with water to replace loss due to evaporation. Potassium sorbate and dye were then added to the aqueous phase and mixed thoroughly.

B) The film-forming ingredients, xanthan gum, locust bean gum, carrageenan and pullulan were mixed together in a separate container to form a film forming mixture.

C) The film forming mixture was slowly added to the aqueous phase of A), followed by overnight mixing at a slow rate to provide a hydrated polymer gel.

D) The flavorants, glycerine, menthol, polysorbate 80 and Atmos 300 were combined and mixed to dissolve in a separate container to yield an organic phase.

E) Mannitol and sucralose were mixed together in the remaining 10% water in a separate container. Succulence was then added to the resulting mixture and dissolved.

F) The mixtures of steps D) and E) were added to the hydrated polymer gel and mixed uniformly to yield a final polymer gel mixture. The final polymer gel mixture was poured on a mold and cast to form a film of a desired thickness at room temperature. The film was dried under warm air and cut to a desired dimension (dictated by e.g., dosage and mouthfeel).

TABLE 1

| | | % w/w* | % w/w | |
|---|---|---|---|---|
| Material | mg/dose* | Dry Film | Actual Batch | g/batch |
| Dextromethorphan | 15.0000 | 22.7322 | 7.7289 | 38.6447 |
| HBr | | | | |
| Amberlite IRP69 | 16.0000 | 24.2477 | 8.2442 | 41.2211 |
| Xanthan Gum | 0.0769 | 0.1165 | 0.0396 | 0.1981 |
| Locust Bean Gum | 0.0901 | 0.1365 | 0.0464 | 0.2321 |
| Carrageenan | 0.3861 | 0.5851 | 0.1989 | 0.9947 |
| Pullulan | 20.5919 | 31.2066 | 10.6102 | 53.0512 |
| Potassium sorbate | 0.0772 | 0.1170 | 0.0398 | 0.1989 |
| Purified water | — | — | 66.0000 | 330.0000 |
| Menthol | 2.5740 | 3.9008 | 1.3263 | 6.6314 |
| Peppermint Flavor | 0.2579 | 0.3908 | 0.1329 | 0.6644 |
| Cherry Flavor | 0.2579 | 0.3908 | 0.1329 | 0.6644 |
| (Givudan) | | | | |
| Sour Cherry (IFF) | 2.2350 | 3.3871 | 1.1516 | 5.7581 |
| Warm Sensation | 0.5518 | 0.8362 | 0.2843 | 1.4216 |
| (Mane) | | | | |
| Artificial Masking | 0.4139 | 0.6273 | 0.2133 | 1.0663 |
| Agent Flavor | | | | |
| (Robertet) | | | | |
| Succulence (IFF) | 0.2579 | 0.3908 | 0.1329 | 0.6644 |
| FD&C Red #40 | 0.0098 | 0.0149 | 0.0050 | 0.0252 |
| Polysorbate 80 NF | 0.4504 | 0.6826 | 0.2321 | 1.1604 |
| Atmos 300 | 0.4504 | 0.6826 | 0.2321 | 1.1604 |
| Glycerine | 1.9305 | 2.9256 | 0.9947 | 4.9736 |
| Mannitol USP | 2.5740 | 3.9008 | 1.3263 | 6.6314 |
| Sucralose | 1.8000 | 2.7279 | 0.9275 | 4.6374 |
| Total | 65.9857 | 100.0000 | 100.0000 | 500.0000 |

*Assuming that all water is evaporated

### EXAMPLE 2

The ingredients listed in Table 2 were combined to provide a consumable film of the present invention in accordance with the following procedure:

A) Dextromethorphan HBr was mixed and dissolved in 90% water at 75° C. to yield an aqueous phase. Amberlite IRP64 was added to the aqueous phase and stirred for about 4 to 5 hours at about 70° C. to 80° C. Pectin was mixed with glycerine and the mixture was added very slowly to the aqueous phase and then mixed thoroughly at a high rate. The aqueous phase was allowed to cool to about 50° C. and q.s. with water to replace loss due to evaporation. Potassium sorbate and dye were then added to the aqueous phase and mixed thoroughly.

B) The film-forming ingredients, xanthan gum, locust bean gum, carrageenan and pullulan were mixed together in a separate container to form a film forming mixture.

C) The film forming mixture was slowly added to the aqueous phase of A), followed by overnight mixing at a slow rate to provide a hydrated polymer gel.

D) The flavorants and menthol were combined and mixed to dissolve in a separate container to yield an organic phase.

E) Mannitol and sucralose were mixed together in the remaining 10% water in a separate container. Succulence was then added to the resulting mixture and dissolved.

F) The mixtures of steps D) and E) were added to the hydrated polymer gel and mixed uniformly to yield a final polymer gel mixture. The final polymer gel mixture was poured on a mold and cast to form a film of a desired thickness at room temperature. The film was dried under warm air and cut to a desired dimension (dictated by e.g., dosage and mouthfeel).

TABLE 2

| | | % w/w* | % w/w | |
|---|---|---|---|---|
| Material | mg/dose* | Dry Film | Actual Batch | g/batch |
| Dextromethorphan | 15.0000 | 22.9235 | 7.8353 | 39.1765 |
| HBr | | | | |
| Amberlite IRP64 | 16.0000 | 24.4518 | 8.3576 | 41.7882 |
| Pectin USP | 0.3500 | 0.5349 | 0.1828 | 0.9141 |
| Xanthan Gum | 0.0769 | 0.1175 | 0.0402 | 0.2008 |
| Locust Bean Gum | 0.0901 | 0.1377 | 0.0471 | 0.2353 |
| Carrageenan | 0.3861 | 0.5901 | 0.2017 | 1.0084 |
| Pullulan | 20.5919 | 31.4693 | 10.7562 | 53.7812 |
| Potassium sorbate | 0.0772 | 0.1180 | 0.0403 | 0.2016 |
| Purified water | — | — | 65.8199 | 329.0995 |
| Menthol | 2.5740 | 3.9337 | 1.3445 | 6.7227 |
| Peppermint Flavor | 0.2579 | 0.3941 | 0.1347 | 0.6736 |
| Cherry Flavor | 0.2579 | 0.3941 | 0.1347 | 0.6736 |
| (Givudan) | | | | |
| Sour Cherry (IFF) | 2.2350 | 3.4156 | 1.1675 | 5.8373 |
| Warm Sensation | 0.5518 | 0.8433 | 0.2882 | 1.4412 |
| (Mane) | | | | |
| Artificial Masking | 0.4139 | 0.6325 | 0.2162 | 1.0810 |
| Agent Flavor | | | | |
| (Robertet) | | | | |
| Succulence (IFF) | 0.2579 | 0.3941 | 0.1347 | 0.6736 |
| FD&C Red #40 | 0.0098 | 0.0150 | 0.0051 | 0.0256 |
| Glycerine | 1.9305 | 2.9503 | 1.0084 | 5.0420 |
| Mannitol USP | 2.5740 | 3.9337 | 1.3445 | 6.7227 |
| Sucralose | 1.8000 | 2.7508 | 0.9402 | 4.7012 |
| Total | 65.4349 | 100.0000 | 100.0000 | 500.0000 |

*Assuming that all water is evaporated

### EXAMPLE 3

The ingredients listed in Table 3 were combined to provide a consumable film of the present invention in accordance with the following procedure:

A) Dextromethorphan HBr was mixed and dissolved in 90% water at 75° C. to yield an aqueous phase. Amberlite IRP69 was added to the aqueous phase and stirred for about 4 to 5 hours at about 70° C. to 80° C. Pectin was added to the aqueous phase very slowly and mixed at high speed. The aqueous phase was allowed to cool to about 50° C. and q.s. with water to replace loss due to evaporation. Potassium sorbate, sweeteners and dye were then added to the aqueous phase and mixed thoroughly.

B) The film-forming ingredients, xanthan gum, locust bean gum, carrageenan and pullulan were mixed together in a separate container to form a film forming mixture.

C) The film forming mixture was slowly added to the aqueous phase of A), followed by overnight mixing at a slow rate to provide a hydrated polymer gel.

D) The flavorants, glycerine, menthol, and surfactants were combined and mixed to dissolve in a separate container to yield an organic phase.

E) Mannitol was mixed together in the remaining 10% water in a separate container. Succulence was then added to the resulting mixture and dissolved.

F) The mixtures of steps D) and E) were added to the hydrated polymer gel and mixed uniformly to yield a final polymer gel mixture. The final polymer gel mixture was poured on a mold and cast to form a film of a desired thickness at room temperature. The film was dried under warm air and cut to a desired dimension (dictated by e.g., dosage and mouthfeel).

TABLE 3

| | | % w/w* | % w/w | |
|---|---|---|---|---|
| Material | mg/dose* | Dry Film | Actual Batch | g/batch |
| Dextromethorphan | 15.0000 | 22.6123 | 7.7289 | 38.6445 |
| HBr | | | | |
| Amberlite IRP69 | 16.0000 | 24.1197 | 8.2442 | 41.2208 |
| Pectin USP | 0.3500 | 0.5276 | 0.1803 | 0.9017 |
| Xanthan Gum | 0.0769 | 0.1159 | 0.0396 | 0.1981 |
| Locust Bean Gum | 0.0901 | 0.1358 | 0.0464 | 0.2321 |
| Carrageenan | 0.3861 | 0.5820 | 0.1989 | 0.9947 |
| Pullulan | 20.5919 | 31.0420 | 10.6102 | 53.0509 |
| Potassium sorbate | 0.0772 | 0.1164 | 0.0398 | 0.1989 |
| Purified water | — | — | 65.8199 | 329.0995 |
| Menthol | 2.5740 | 3.8803 | 1.3263 | 6.6314 |
| Peppermint Flavor | 0.2579 | 0.3888 | 0.1329 | 0.6644 |
| Cherry Flavor | 0.2579 | 0.3888 | 0.1329 | 0.6644 |
| (Givudan) | | | | |
| Cherry Flavor Blend | 2.2350 | 3.3692 | 1.1516 | 5.7580 |
| (IFF) | | | | |
| Warm Sensation | 0.5518 | 0.8318 | 0.2843 | 1.4216 |
| (Mane) | | | | |
| Artificial Masking | 0.4139 | 0.6239 | 0.2133 | 1.0663 |
| Agent | | | | |
| Flavor (Robertet) | | | | |
| Succulence (IFF) | 0.2579 | 0.3888 | 0.1329 | 0.6644 |
| FD&C Red #40 | 0.0098 | 0.0148 | 0.0050 | 0.0252 |
| Polysorbate 80 NF | 0.4504 | 0.6790 | 0.2321 | 1.1604 |
| Atmos 300 | 0.4504 | 0.6790 | 0.2321 | 1.1604 |
| Glycerine | 1.9305 | 2.9102 | 0.9947 | 4.9735 |
| Mannitol USP | 2.5740 | 3.8803 | 1.3263 | 6.6314 |
| Sucralose | 1.8000 | 2.7135 | 0.9275 | 4.6373 |
| Total | 66.3357 | 100.0000 | 100.0000 | 500.0000 |

*Assuming that all water is evaporated

### EXAMPLE 4

The ingredients listed in Table 4 were combined to provide a consumable film of the present invention in accordance with the following procedure:

A) Dextromethorphan HBr was mixed and dissolved in 90% water at 75° C. to yield an aqueous phase. Amberlite IRP64 was added to the aqueous phase and stirred for about 4 to 5 hours at about 70° C. to 80° C. Pectin was added to the aqueous phase very slowly and mixed at a high mixing rate. The aqueous phase was allowed to cool to about 50° C. and q.s. with water to replace loss due to evaporation. Potassium sorbate and dye were then added to the aqueous phase and mixed thoroughly.

B) The film-forming ingredients, xanthan gum, locust bean gum, carrageenan and pullulan were mixed together in a separate container to form a film forming mixture.

C) The film forming mixture was slowly added to the aqueous phase of A), followed by overnight mixing at a low mixing rate to provide a hydrated polymer gel.

D) The flavorants, glycerine, menthol, and surfactants were combined and mixed to dissolve in a separate container to yield an organic phase.

E) Mannitol and sucralose were mixed together in the remaining 10% water in a separate container. Succulence was then added to the resulting mixture and dissolved.

F) The mixtures of steps D) and E) were added to the hydrated polymer gel and mixed uniformly to yield a final polymer gel mixture. The final polymer gel mixture was poured on a mold and cast to form a film of a desired thickness at room temperature. The film was dried under warm air and cut to a desired dimension (dictated by e.g., dosage and mouthfeel).

TABLE 4

| | | % w/w* | % w/w | |
|---|---|---|---|---|
| Material | mg/dose* | Dry Film | Actual Batch | g/batch |
| Dextromethorphan | 15.0000 | 22.6123 | 7.7289 | 38.6445 |
| HBr | | | | |
| Amberlite IRP64 | 16.0000 | 24.1197 | 8.2442 | 41.2208 |
| Pectin USP | 0.3500 | 0.5276 | 0.1803 | 0.9017 |
| Xanthan Gum | 0.0769 | 0.1159 | 0.0396 | 0.1981 |
| Locust Bean Gum | 0.0901 | 0.1358 | 0.0464 | 0.2321 |
| Carrageenan | 0.3861 | 0.5820 | 0.1989 | 0.9947 |
| Pullulan | 20.5919 | 31.0420 | 10.6102 | 53.0509 |
| Potassium sorbate | 0.0772 | 0.1164 | 0.0398 | 0.1989 |
| Purified water | — | — | 65.8199 | 329.0995 |
| Menthol | 2.5740 | 3.8803 | 1.3263 | 6.6314 |
| Peppermint Flavor | 0.2579 | 0.3888 | 0.1329 | 0.6644 |
| Cherry Flavor | 0.2579 | 0.3888 | 0.1329 | 0.6644 |
| (Givudan) | | | | |
| Sour Cherry (IFF) | 2.2350 | 3.3692 | 1.1516 | 5.7580 |
| Warm Sensation | 0.5518 | 0.8318 | 0.2843 | 1.4216 |
| (Mane) | | | | |
| Artificial Masking | 0.4139 | 0.6239 | 0.2133 | 1.0663 |
| Agent Flavor | | | | |
| (Robertet) | | | | |
| Succulence (IFF) | 0.2579 | 0.3888 | 0.1329 | 0.6644 |
| FD&C Red #40 | 0.0098 | 0.0148 | 0.0050 | 0.0252 |
| Polysorbate 80 NF | 0.4504 | 0.6790 | 0.2321 | 1.1604 |
| Atmos 300 | 0.4504 | 0.6790 | 0.2321 | 1.1604 |
| Glycerine | 1.9305 | 2.9102 | 0.9947 | 4.9735 |
| Mannitol USP | 2.5740 | 3.8803 | 1.3263 | 6.6314 |
| Sucralose | 1.8000 | 2.7135 | 0.9275 | 4.6373 |
| Total | 66.3357 | 100.0000 | 100.0000 | 500.0000 |

*Assuming that all water is evaporated

### EXAMPLE 5

The ingredients listed in Table 5 were combined to provide a consumable film of the present invention in accordance with the following procedure:

A) Dextromethorphan HBr was mixed and dissolved in 90% water at 75° C. to yield an aqueous phase. Amberlite IRP69 was added to the aqueous phase and stirred for about 4 to 5 hours at about 70° C. to 80° C. Pectin was added to the aqueous phase very slowly and mixed at a high mixing rate. The aqueous phase was allowed to cool to about 50° C. and q.s. with water to replace loss due to evaporation. Potassium sorbate and dye were then added to the aqueous phase and mixed thoroughly.

B) The film-forming ingredients, xanthan gum, locust bean gum, carrageenan and PURE-COTE B793 were mixed together in a separate container to form a film forming mixture.

C) The film forming mixture was slowly added to the aqueous phase of A), followed by overnight mixing at a low mixing rate to provide a hydrated polymer gel.

D) The flavorants, glycerine, olive oil, menthol, and surfactants were combined and mixed to dissolve in a separate container to yield an organic phase.

E) Mannitol and sucralose were mixed together in the remaining 10% water in a separate container. Succulence was then added to the resulting mixture and dissolved.

F) The mixtures of steps D) and E) were added to the hydrated polymer gel and mixed uniformly to yield a final polymer gel mixture. The final polymer gel mixture was poured on a mold and cast to form a film of a desired thickness at room temperature. The film was dried under warm air and cut to a desired dimension (dictated by e.g., dosage and mouthfeel).

TABLE 5

| | | % w/w* | % w/w | |
|---|---|---|---|---|
| Material | mg/dose* | Dry Film | Actual Batch | g/batch |
| Dextromethorphan | 15.0000 | 19.5740 | 10.6759 | 106.7593 |
| HBr | | | | |
| Amberlite IRP69 | 16.0001 | 20.8790 | 11.3877 | 113.8771 |
| Pectin USP | 0.3499 | 0.4566 | 0.2490 | 2.4905 |
| Xanthan Gum | 0.0769 | 0.1003 | 0.0547 | 0.5470 |
| Locust Bean Gum | 0.0901 | 0.1175 | 0.0641 | 0.6409 |
| Carrageenan | 0.3860 | 0.5037 | 0.2747 | 2.7474 |
| PURE-COTE B793 | 20.5919 | 26.8711 | 14.6559 | 146.5586 |
| Potassium sorbate | 0.0772 | 0.1008 | 0.0550 | 0.5498 |
| Purified water | — | — | 45.4586 | 454.5856 |
| Menthol | 2.5740 | 3.3589 | 1.8320 | 18.3202 |
| Peppermint Flavor | 0.2579 | 0.3366 | 0.1836 | 1.8357 |
| Cherry Flavor | 0.2579 | 0.3366 | 0.1836 | 1.8357 |
| (Givudan) | | | | |
| Sour Cherry (IFF) | 2.2350 | 2.9165 | 1.5907 | 15.9070 |
| Warm Sensation | 0.5518 | 0.7200 | 0.3927 | 3.9270 |
| (Mane) | | | | |
| Artificial Masking | 0.4140 | 0.5402 | 0.2946 | 2.9463 |
| Agent Flavor | | | | |
| (Robertet) | | | | |
| Succulence (IFF) | 0.2579 | 0.3366 | 0.1836 | 1.8357 |
| FD&C Red #40 | 0.0099 | 0.0129 | 0.0070 | 0.0704 |
| Polysorbate 80 NF | 0.4505 | 0.5878 | 0.3206 | 3.2060 |
| Atmos 300 | 0.4505 | 0.5878 | 0.3206 | 3.2060 |
| Glycerine | 8.7335 | 11.3966 | 6.2158 | 62.1585 |
| Olive Oil | 3.49934 | 4.5586 | 2.4863 | 24.8634 |
| Mannitol USP | 2.5740 | 3.3589 | 1.8320 | 18.3202 |
| Sucralose | 1.8001 | 2.3490 | 1.2812 | 12.8116 |
| Total | 76.6324 | 100.0000 | 100.0000 | 1000.0000 |

*Assuming that all water is evaporated

### EXAMPLE 6

The ingredients listed in Table 6 were combined to provide a consumable film of the present invention in accordance with the procedure example 5 with Amberlite IRP64 being substituted by Amberlite IRP69.

TABLE 6

| | | % w/w* | % w/w | |
|---|---|---|---|---|
| Material | mg/dose* | Dry Film | Actual Batch | g/batch |
| Dextromethorphan | 15.0000 | 18.5409 | 10.3611 | 103.6107 |
| HBr | | | | |
| Amberlite IRP69 | 16.0001 | 19.7771 | 11.0519 | 110.5186 |
| Pectin USP | 0.3499 | 0.4325 | 0.2417 | 2.4170 |
| Xanthan Gum | 0.0769 | 0.0950 | 0.0531 | 0.5309 |
| Locust Bean Gum | 0.0901 | 0.1113 | 0.0622 | 0.6220 |
| Carrageenan | 0.3860 | 0.4771 | 0.2666 | 2.6664 |
| PURE-COTE B793 | 20.5919 | 25.4529 | 14.2236 | 142.2363 |
| Potassium sorbate | 0.0772 | 0.0955 | 0.0534 | 0.5335 |
| Purified water | — | — | 44.1179 | 451.1788 |
| Menthol | 2.5740 | 3.1817 | 1.7780 | 17.7799 |
| Peppermint Flavor | 0.2579 | 0.3188 | 0.1782 | 1.7816 |
| Cherry Flavor | 0.2579 | 0.3188 | 0.1782 | 1.7816 |
| (Givudan) | | | | |
| Sour Cherry (IFF) | 2.2350 | 2.7626 | 1.5438 | 15.4379 |
| Warm Sensation | 0.5518 | 0.6820 | 0.3811 | 3.8112 |
| (Mane) | | | | |
| Artificial Masking | 0.4140 | 0.5117 | 0.2859 | 2.8594 |
| Agent Flavor | | | | |
| (Robertet) | | | | |
| Succulence (IFF) | 0.2579 | 0.3188 | 0.1782 | 1.7816 |
| FD&C Red #40 | 0.0099 | 0.0122 | 0.0068 | 0.0684 |
| Polysorbate 80 NF | 0.4505 | 0.5568 | 0.3111 | 3.1114 |
| Atmos 300 | 0.4505 | 0.5568 | 0.3111 | 3.1114 |
| Glycerine | 11.6446 | 14.3935 | 8.0434 | 80.4337 |
| Olive Oil | 4.8519 | 5.9973 | 3.3514 | 33.5140 |
| Mannitol USP | 2.5740 | 3.1817 | 1.7780 | 17.7799 |
| Sucralose | 1.8001 | 2.2250 | 1.2434 | 12.4337 |
| Total | 80.9021 | 100.0000 | 100.0000 | 1000.0000 |

Assuming that all water is evaporated

### EXAMPLE 7

The ingredients listed in Table 7 were combined to provide a consumable film of the present invention in accordance with the following procedure:

A) Dextromethorphan HBr was mixed and dissolved in 90% water at 75° C. to yield an aqueous phase. Amberlite IRP69 was added to the aqueous phase and stirred for about 4 to 5 hours at about 70° C. to 80° C. Pectin dispersed in glycerine was added very slowly to the aqueous phase and mixed at a high mixing rate. The aqueous phase was allowed to cool to about 50° C. and q.s. with water to replace loss due to evaporation. The dye was then added to the aqueous phase and mixed thoroughly.

B) The film-forming ingredients, xanthan gum, locust bean gum, carrageenan and pullulan were mixed together in a separate container to form a film forming mixture.

C) The film forming mixture was slowly added to the aqueous phase of A), followed by overnight mixing at a low mixing rate to provide a hydrated polymer gel.

D) The flavorants, menthol, and surfactants were combined and mixed to dissolve in a separate container to yield an organic phase.

E) Mannitol and sucralose were mixed together in the remaining 10% water in a separate container. Succulence was then added to the resulting mixture and dissolved.

F) The mixtures of steps D) and E) were added to the hydrated polymer gel and mixed uniformly to yield a final polymer gel mixture. The final polymer gel mixture was poured on a mold and cast to form a film of a desired thickness at room temperature. The film was dried under warm air and cut to a desired dimension (dictated by e.g., dosage and mouthfeel).

TABLE 7

| | | % w/w* | % w/w | |
|---|---|---|---|---|
| Material | mg/dose* | Dry Film | Actual Batch | g/batch |
| Dextromethorphan | 15.0000 | 22.5510 | 7.7080 | 19.2699 |
| HBr | | | | |
| Amberlite IRP64 | 16.0000 | 24.0544 | 8.2218 | 20.5545 |
| Pectin USP | 0.3500 | 0.5262 | 0.1799 | 0.4496 |
| Xanthan Gum | 0.0769 | 0.1156 | 0.0395 | 0.0988 |
| Locust Bean Gum | 0.0901 | 0.1355 | 0.0463 | 0.1157 |
| Carrageenan | 0.3861 | 0.5805 | 0.1984 | 0.4960 |
| Pullulan | 20.5919 | 30.9579 | 10.5814 | 26.4536 |
| Potassium sorbate | 0.0772 | 0.1161 | 0.0397 | 0.0992 |
| Purified water | — | — | 65.8199 | 164.5498 |
| Menthol | 2.5740 | 3.8698 | 1.3227 | 3.3067 |
| Peppermint Flavor | 0.2579 | 0.3877 | 0.1325 | 0.3313 |
| Cherry Flavor | 0.2579 | 0.3877 | 0.1325 | 0.3313 |
| (Givudan) | | | | |
| Sour Cherry (IFF) | 2.2350 | 3.3601 | 1.1485 | 2.8712 |
| Warm Sensation | 0.5518 | 0.8296 | 0.2835 | 0.7089 |
| (Mane) | | | | |
| Artificial Masking | 0.4139 | 0.6223 | 0.21 27 | 0.5317 |
| Agent Flavor | | | | |
| (Robertet) | | | | |
| Succulence (IFF) | 0.2579 | 0.3877 | 0.1325 | 0.3313 |
| Carmine | 0.1900 | 0.2856 | 0.0976 | 0.2441 |
| Polysorbate 80 NF | 0.4504 | 0.6771 | 0.2314 | 0.5786 |
| Atsurf 596K | 0.4504 | 0.6771 | 0.2314 | 0.5786 |
| Glycerine | 1.9305 | 2.9023 | 0.9920 | 2.4800 |
| Mannitol USP | 2.5740 | 3.8698 | 1.3227 | 3.3067 |
| Sucralose | 1.8000 | 2.7061 | 0.9250 | 2.3124 |
| Total | 66.5159 | 100.0000 | 100.0000 | 250.0000 |

*Assuming that all water is evaporated

### EXAMPLE 8

The ingredients listed in Table 8 were combined to provide a consumable film of the present invention in accordance with the procedure described in Example 7.

TABLE 8

| | | % w/w* | % w/w | |
|---|---|---|---|---|
| Material | mg/dose* | Dry Film | Actual Batch | g/batch |
| Dextromethorphan | 15.0000 | 22.5772 | 7.7169 | 38.5846 |
| HBr | | | | |
| Amberlite IRP64 | 16.0000 | 24.0823 | 8.2314 | 41.1569 |
| Pectin USP | 0.3500 | 0.5268 | 0.1801 | 0.9003 |
| Xanthan Gum | 0.0769 | 0.1157 | 0.0396 | 0.1978 |
| Locust Bean Gum | 0.0901 | 0.1356 | 0.0464 | 0.2318 |
| Carrageenan | 0.3861 | 0.5811 | 0.1986 | 0.9932 |
| Pullulan | 20.5919 | 30.9938 | 10.5937 | 52.9686 |
| Carmine | 0.1900 | 0.2860 | 0.0977 | 0.4887 |
| Purified water | — | — | 65.8199 | 329.0995 |
| Menthol | 2.5740 | 3.8742 | 1.3242 | 6.6211 |
| Peppermint Flavor | 0.2579 | 0.3882 | 0.1327 | 0.6634 |
| Cherry Flavor | 0.2579 | 0.3882 | 0.1327 | 0.6634 |
| (Givudan) | | | | |
| Sour Cherry (IFF) | 2.2350 | 3.3640 | 1.1498 | 5.7491 |
| Warm Sensation | 0.5518 | 0.8305 | 0.2839 | 1.4194 |
| (Mane) | | | | |
| Artificial Masking | 0.4139 | 0.6230 | 0.2129 | 1.0647 |
| Agent | | | | |
| Flavor (Robertet) | | | | |
| Succulence (IFF) | 0.2579 | 0.3882 | 0.1327 | 0.6634 |
| Polsorbate 80 NF | 0.4504 | 0.6779 | 0.2317 | 1.1586 |
| Atmos 300 | 0.4504 | 0.6779 | 0.2317 | 1.1586 |
| Glycerine | 1.9305 | 2.9057 | 0.9932 | 4.9658 |
| Mannitol USP | 2.5740 | 3.8742 | 1.3242 | 6.6211 |
| Sucralose | 1.8000 | 2.7093 | 0.9260 | 4.6301 |
| Total | 66.4387 | 100.0000 | 100.0000 | 500.0000 |

*Assuming that all water is evaporated

### EXAMPLE 9

The ingredients listed in Table 9 were combined to provide a consumable film of the present invention in accordance with the procedure described in Example 7 absent the resin.

TABLE 9

| | | % w/w* | % w/w | |
|---|---|---|---|---|
| Material | mg/dose* | Dry Film | Actual Batch | g/batch |
| Dextromethorphan | 10.9900 | 18.3460 | 5.5038 | 27.5189 |
| (Spectrum) | | | | |
| Pectin USP | 0.5250 | 0.8764 | 0.2629 | 1.3146 |
| Carmine | 0.1900 | 0.3172 | 0.0952 | 0.4758 |
| Xanthan Gum | 0.1154 | 0.1926 | 0.0578 | 0.2888 |
| Locust Bean Gum | 0.1352 | 0.2256 | 0.0677 | 0.3384 |
| Carrageenan | 0.5792 | 0.9668 | 0.2900 | 1.4502 |
| Pullulan | 30.8879 | 51.5621 | 15.4686 | 77.3431 |
| Purified water | — | — | 70 | 350.0000 |
| Menthol | 2.5740 | 4.2969 | 1.2891 | 6.4453 |
| Peppermint Flavor | 0.8000 | 1.3355 | 0.4006 | 2.0032 |
| Cherry Flavor | 0.8000 | 1.3355 | 0.4006 | 2.0032 |
| (Givudan) | | | | |
| Sour Cherry (IFF) | 2.2350 | 3.7310 | 1.1193 | 5.5964 |
| Warm Sensation | 0.8000 | 1.3355 | 0.4006 | 2.0032 |
| (Mane) | | | | |
| Artificial Masking | 0.8000 | 1.3355 | 0.4006 | 2.0032 |
| Agent Flavor | | | | |
| (Robertet) | | | | |
| Succulence (IFF) | 0.2579 | 0.4305 | 0.1292 | 0.6458 |
| Polysorbate 80 NF | 0.4504 | 0.7519 | 0.2256 | 1.1278 |
| Atmos 300 | 0.4504 | 0.7519 | 0.2256 | 1.1278 |
| Glycerine | 2.0400 | 3.4054 | 1.0216 | 5.1082 |
| Sucralose | 2.7000 | 4.5072 | 1.3522 | 6.7608 |
| Mannitol USP | 2.5740 | 4.2969 | 1.2891 | 6.4453 |
| Total | 59.9042 | 100.0000 | 100.0000 | 500.0000 |

*Assuming that all water is evaporated

### EXAMPLE 10

The ingredients listed in Table 10 were combined to provide a consumable film of the present invention in accordance with the procedure described in Example 7.

TABLE 10

| | | % w/w* | % w/w | |
|---|---|---|---|---|
| Material | mg/dose* | Dry Film | Actual Batch | g/batch |
| Dextromethorphan | 10.9900 | 26.6157 | 9.2695 | 18.5390 |
| (milled) | | | | |
| Amberlite IRP69 | 2.4000 | 5.8123 | 2.0243 | 4.04486 |
| Pectin USP | 0.2698 | 0.6534 | 0.2276 | 0.4551 |
| Carmine | 0.1464 | 0.3546 | 0.1235 | 0.2470 |
| Xanthan Gum | 0.0594 | 0.1439 | 0.0501 | 0.1002 |
| Locust Bean Gum | 0.0694 | 0.1681 | 0.0585 | 0.1171 |
| Carrageenan | 0.2975 | 0.7205 | 0.2509 | 0.5019 |
| Pullulan | 15.8694 | 38.4327 | 13.3850 | 26.7701 |
| Purified water | — | — | 65.1728 | 130.3456 |
| Menthol | 2.5740 | 6.2337 | 2.1710 | 4.3421 |
| Peppermint Flavor | 0.1987 | 0.4812 | 0.1676 | 0.3352 |
| Cherry Flavor | 0.1987 | 0.4812 | 0.1676 | 0.3352 |
| (Givudan) | | | | |
| Sour Cherry (IFF) | 1.7225 | 4.1716 | 1.4528 | 2.9057 |
| Warm Sensation | 0.4252 | 1.0298 | 0.3586 | 0.7173 |
| (Mane) | | | | |
| Artificial Masking | 0.3190 | 0.7726 | 0.2691 | 0.5381 |
| Agent Flavor | | | | |
| (Robertet) | | | | |
| Succulence IFF | 0.1987 | 0.4812 | 0.1676 | 0.3352 |
| Polysorbate 80 NF | 0.3470 | 0.8404 | 0.2927 | 0.5854 |
| Atmos 300 | 0.3470 | 0.8404 | 0.2927 | 0.5854 |
| Glycerine | 1.4877 | 3.6029 | 1.2548 | 2.5096 |
| Mannitol USP | 1.9837 | 4.8041 | 1.6732 | 3.3463 |
| Sucralose | 1.3873 | 3.3598 | 1.1701 | 2.3402 |
| Total | 41.2914 | 100.0000 | 100.0000 | 200.0000 |

*Assuming that all water is evaporated

### EXAMPLE 11

The ingredients listed in Table 11 were combined to provide a consumable film of the present invention in accordance with the following procedure:

A) Potassium sorbate and dye were mixed in 80% water.

B) The film-forming ingredients, xanthan gum, locust bean gum, carrageenan and Pure-Cote B793 were mixed together in a separate container to form a film forming mixture.

C) The film-forming mixture was slowly added to the mixture of A), followed by overnight mixing at a low mixing rate to form a hydrated polymer gel.

D) Mannitol and sucralose were mixed together with remaining 20% of water in a separate container, and then added to the hydrated polymer gel and mixed well.

E) Milled famotidine HCl was added to the hydrated polymer gel and mixed thoroughly.

F) The flavorants, glycerine, olive oil and surfactants were combined and mixed thoroughly in a separate container.

G) The resulting mixture of step F) was added to the hydrated polymer gel and mixed uniformly to yield a final polymer gel mixture. The final polymer gel mixture was poured on a mold and cast to form a film of a desired thickness at room temperature. The film was dried under warm air and cut to a desired dimension (dictated by e.g., dosage and mouthfeel).

TABLE 11

| | | % w/w* | % w/w | |
|---|---|---|---|---|
| Material | mg/dose* | Dry Film | Actual Batch | g/batch |
| Famotidine | 10.0000 | 15.2065 | 5.3223 | 106.4453 |
| Xanthan Gum | 0.1154 | 0.1754 | 0.0614 | 1.2278 |
| Locust Bean Gum | 0.1352 | 0.2055 | 0.0719 | 1.4386 |
| Carrageenan | 0.5792 | 0.8807 | 0.3082 | 6.1648 |
| Pure-Cote B793 | 30.8879 | 46.9695 | 16.4393 | 328.7865 |
| Potassium sorbate | 0.1158 | 0.1761 | 0.0616 | 1.2326 |
| Purified water | — | — | 65.0000 | 1300.0000 |
| Vanilla Mint Flavor | 2.0000 | 3.0413 | 1.0645 | 21.2891 |
| (IFF) | | | | |
| Polysorbate 80 NF | 0.6756 | 1.0273 | 0.3596 | 7.1914 |
| Atsurf 596K | 0.6756 | 1.0273 | 0.3596 | 7.1914 |
| Glycerine | 10.0000 | 15.2065 | 5.3223 | 106.4453 |
| Olive oil | 4.0000 | 6.0826 | 2.1289 | 42.5781 |
| FD&C Blue #1 | 0.0160 | 0.0243 | 0.0085 | 0.1703 |
| Mannitol USP | 3.8610 | 5.8712 | 2.0549 | 41.0985 |
| Sucralose | 2.7000 | 4.1057 | 1.4370 | 28.7402 |
| Total | 65.7615 | 100.0000 | 100.0000 | 2000.0000 |

*Assuming that all water is evaporated

### EXAMPLE 12

The ingredients listed in Table 12 were combined to provide a consumable film of the present invention in accordance with the procedure described in Example 11 with the PURE-COTE B793 substituted by Tapioca Starch J474.

TABLE 12

| | | % w/w* | % w/w* | |
|---|---|---|---|---|
| Material | mg/dose* | Dry Film | Actual Batch | g/batch |
| Famotidine | 10.0000 | 9.7503 | 4.4512 | 26.6184 |
| Xanthan Gum | 0.1154 | 0.1125 | 0.0513 | 0.3070 |
| Locust Bean Gum | 0.1352 | 0.1318 | 0.0602 | 0.3597 |
| Carrageenan | 0.5792 | 0.5647 | 0.2578 | 1.5416 |
| Tapioca Starch J474 | 67.6870 | 65.9970 | 30.1291 | 180.1720 |
| Potassium sorbate | 0.1158 | 0.1129 | 0.0515 | 0.3082 |
| Purified water | — | — | 54.3478 | 324.9998 |
| Vanilla Mint Flavor | 2.0000 | 1.9501 | 0.8902 | 5.237 |
| (IFF) | | | | |
| Polysorbate 80 NF | 0.6756 | 0.6587 | 0.3007 | 1.7983 |
| Atsurf 596K | 0.6756 | 0.6587 | 0.3007 | 1.7983 |
| Glycerine | 10.0000 | 9.7503 | 4.4512 | 26.6184 |
| Olive oil | 4.0000 | 3.9001 | 1.7805 | 10.6474 |
| FD&C Blue #1 | 0.0160 | 0.0156 | 0.0071 | 0.0426 |
| Mannitol USP | 3.8610 | 3.7646 | 1.7186 | 10.2774 |
| Sucralose | 2.7000 | 2.6326 | 1.2018 | 7.1870 |
| Total | 102.5607 | 100.0000 | 100.0000 | 598.0000 |

*Assuming that all water is evaporated

### EXAMPLE 13

The ingredients listed in Table 13 were combined to provide a consumable film of the present invention in accordance with the following procedure:

A) The dye, copper gluconate, acesulfame potassium salt, and aspartame were dissolved in water and mixed for about 30 minutes to yield an aqueous phase.

B) The film-forming ingredients, xanthan gum, locust bean gum, carrageenan and pullulan were mixed together in a separate container to form a film forming mixture.

C) The film forming mixture was slowly added to the aqueous phase of A), followed by overnight mixing at a low mixing rate to provide a hydrated polymer emulsion.

D) The mint flavor, Physcool, thymol, methyl salicylate, eucalyptol and menthol were combined and mixed to dissolve in a separate container to yield an organic phase.

E) The organic phase was added to the hydrated polymer emulsion and mixed uniformly to yield a final polymer emulsion mixture. The final polymer emulsion mixture was poured on a mold and cast to form a film of a desired thickness at room temperature. The film was dried under warm air and cut to a desired dimension (dictated by e.g., dosage and mouthfeel).

TABLE 13

| | % w/w* | % w/w | |
|---|---|---|---|
| Material | Dry Film | Actual Batch | g/batch |
| Xanthan Gum | 0.1393 | 0.0344 | 0.1722 |
| Locust Bean Gum | 0.2786 | 0.0689 | 0.3444 |
| Carrageenan | 1.3929 | 0.3444 | 1.7222 |
| Pullulan | 66.9165 | 16.5475 | 82.7374 |
| FD&C Green No. 3 | 0.0106 | 0.0026 | 0.0131 |
| Copper gluconate | 1.4459 | 0.3575 | 1.7877 |
| Acesulfame Potassium | 1.8083 | 0.4472 | 2.2359 |
| Aspartame | 5.7875 | 1.4312 | 7.1558 |
| Purified water | — | 75.2714 | 376.3571 |
| Mint Flavor | 10.8500 | 2.6830 | 13.4152 |
| Physcool/Mint Flavor | 0.3625 | 0.0896 | 0.4482 |
| Thymol | 0.5295 | 0.1309 | 0.6546 |
| Methyl salicylate | 0.7575 | 0.1873 | 0.9367 |
| Eucalyptol | 0.7575 | 0.1873 | 0.9367 |
| Menthol | 8.9635 | 2.2165 | 11.0827 |
| Total | 100.0000 | 100.0000 | 500.0000 |

*Assuming that all water is evaporated

### EXAMPLE 14

The ingredients listed in Table 14 were combined to provide a consumable film of the present invention in accordance with the following procedure:

A) Dextromethorphan HBr was mixed and dissolved in 90% water at 75° C. to yield an aqueous phase. Sodium bicarbonate was added and mixed for about 1 hour. Amberlite IRP69 was added to the aqueous phase and stirred for about 2 hours at about 70° C. to 80° C. The resulting mixture was allowed to cool to 50° C. and q.s. with water for losses due to evaporation. The dye was then added to the aqueous phase and mixed thoroughly.

B) The film-forming ingredients, xanthan gum, locust bean gum, carrageenan and pullulan were added slowly and rapidly mixed together in a separate container to form a film forming mixture. The mixture was mixed overnight at a low speed. Pectin dispersed in glycerine was added very slowly to the film forming mixture and mixed at a high mixing rate.

C) The film forming mixture was slowly added to the aqueous phase of A), followed by overnight mixing at a low mixing rate to provide a hydrated polymer gel.

D) In another container the remaining 10% water was added to dissolve mannitol and sucralose. Succulence was then added and mixed to dissolve. The resulting mixture was added to the hydrated polymer gel.

E) The flavorants, menthol, and surfactants were combined and mixed to dissolve in a separate container to yield an organic phase.

F) The mixtures of steps D) and E) were added together and mixed uniformly to yield a final polymer gel mixture. The final polymer gel mixture was poured on a mold and cast to form a film of a desired thickness at room temperature. The film was dried under warm air and cut to a desired dimension (dictated by e.g., dosage and mouthfeel).

TABLE 14

| | | | % w/w* | |
|---|---|---|---|---|
| | | % w/w | Actual | |
| Material | mg/dose* | Dry Film | Batch | g/batch |
| Dextromethorphan | 15.0000 | 27.3219 | 9.6903 | 484.5135 |
| HBr | | | | |
| Amberlite IRP69 | 8.0000 | 14.5717 | 5.1681 | 258.4072 |
| Pectin USP | 0.2698 | 0.4914 | 0.1743 | 8.7148 |
| Sodium bicarbonate | 4.0000 | 7.2858 | 2.5841 | 129.2036 |
| anhydrous | | | | |
| Carmine | 0.1464 | 0.2667 | 0.0946 | 4.7289 |
| Xanthan Gum | 0.0594 | 0.1082 | 0.0384 | 1.91187 |
| Locust Bean Gum | 0.0694 | 0.1264 | 0.0448 | 2.2417 |
| Carrageenan | 0.2975 | 0.5419 | 0.1922 | 9.6095 |
| Pullulan | 15.8690 | 28.9047 | 10.2517 | 512.5830 |
| Purified water | — | — | 64.5329 | 3226.6450 |
| Menthol | 2.5740 | 4.6884 | 1.6629 | 83.1425 |
| Peppermint Flavor | 0.1987 | 0.3619 | 0.1284 | 6.4182 |
| Cherry Flavor | 0.1987 | 0.3619 | 0.1284 | 6.4182 |
| (Givudan) | | | | |
| Cherry Flavor Blend | 1.7225 | 3.1375 | 1.1128 | 55.6383 |
| (IFF) | | | | |
| Warm Sensation | 0.4252 | 0.7745 | 0.2747 | 13.7343 |
| (Mane) | | | | |
| Artificial Masking | 0.3190 | 0.5810 | 0.2061 | 10.3040 |
| Agent Flavor | | | | |
| (Robertet) | | | | |
| Succulence (IFF) | 0.1987 | 0.3619 | 0.1284 | 6.4182 |
| Polysorbate 80 NF | 0.3470 | 0.6320 | 0.2242 | 11.2084 |
| Atmos 300 | 0.3470 | 0.6320 | 0.2242 | 11.2084 |
| Glycerine | 1.4877 | 2.7100 | 0.9611 | 48.0573 |
| Mannitol USP | 1.9837 | 3.6132 | 1.2815 | 64.0753 |
| Sucralose | 1.3873 | 2.5269 | 0.8962 | 44.8110 |
| Total | 54.9011 | 100.0000 | 100.0000 | 50000.0000 |

*Assuming that all water is evaporated

### EXAMPLE 15

The ingredients listed in Table 15 were combined to provide a consumable film of the present invention in accordance with the following procedure:

A) Dextromethorphan HBr was mixed and dissolved in water at 50° C. to yield an aqueous phase. Potassium sorbate and the sweeteners were added to the aqueous phase and stirred. Titanium dioxide was then added and the mixture was further stirred.

B) The film-forming ingredients, xanthan gum, locust bean gum, carrageenan and pullulan were mixed together in a separate container to form a film forming mixture.

C) The film forming mixture was slowly added to the aqueous phase of A) while mixing rapidly. The resulting mixture was mixed overnight at a low mixing rate to provide a hydrated polymer gel.

D) Glycerin and olive oil were mixed in a separate container. Menthol and mono ammonium glycyrrhizinate were added to the glycerin/olive oil mixture and heated to dissolve at 45° C. Physcool, polysorbate 80 and Atmos 300 were then added to the resulting mixture and further mixed to yield an organic phase.

E) The mixture of step D) was added to the hydrated polymer gel and mixed uniformly to yield a final polymer gel mixture. Cherry flavor was then added to the polymer gel mixture. The final polymer gel mixture was poured on a mold and cast to form a film of a desired thickness at room temperature. The film was dried under warm air and cut to a desired dimension (dictated by e.g., dosage and mouthfeel).

TABLE 15

| | | | % w/w | |
|---|---|---|---|---|
| | | % w/w* | Actual | |
| Material | mg/dose* | Dry Film | Batch | g/batch |
| Dextromethorphan Coated | 25.0000 | 27.7778 | 8.4515 | 0.2770 |
| (60% dex) | | | | |
| Xanthan Gum | 0.1625 | 0.1805 | 0.0549 | 0.0018 |
| Locust Bean Gum | 0.1896 | 0.2106 | 0.0641 | 0.0021 |
| Carrageenan | 0.8124 | 0.9027 | 0.2746 | 0.0090 |
| Pullulan | 43.3286 | 48.1429 | 14.6478 | 0.4800 |
| Potassium Sorbate | 0.1625 | 0.1805 | 0.0549 | 0.0018 |
| Acesulfame Potassium Salt | 1.3540 | 1.5045 | 0.4577 | 0.0150 |
| Aspartame NF | 3.7913 | 4.2125 | 1.2817 | 0.0420 |
| Purified water | — | — | 69.5744 | 2.2799 |
| Physcool | 0.2708 | 0.3009 | 0.0915 | 0.0030 |
| Menthol | 2.7080 | 3.0089 | 0.9155 | 0.0300 |
| Cherry Flavor (Givudan) | 0.1354 | 0.1504 | 0.0458 | 0.0015 |
| Mono ammonium | | | | |
| glycyrrhizinate (MAG) | 0.0271 | 0.0301 | 0.0092 | 0.0003 |
| Polysorbate 80 NF | 0.9478 | 1.0531 | 0.3204 | 0.0105 |
| Atmos 300 | 0.9478 | 1.0531 | 0.3204 | 0.0105 |
| Glycerine | 8.1241 | 9.0268 | 2.7465 | 0.0900 |
| Olive Oil | 1.3540 | 1.5045 | 0.4577 | 0.0150 |
| FD&C green #3 | 0.0070 | 0.0078 | 0.0024 | 0.0001 |
| Titanium Dioxide | 0.6770 | 0.7522 | 0.2289 | 0.0075 |
| Total | 90.0000 | 100.0000 | 100.0000 | 3.2770 |

*Assuming that all water is evaporated

### EXAMPLE 16

The ingredients listed in Table 16 were combined to provide a consumable film of the present invention in accordance with the following procedure:

A) Dextromethorphan HBr was mixed and dissolved in water at 75° C. to yield an aqueous phase. Amberlite IRP69 was added to the aqueous phase and stirred for about 2 hours at about 70° C. to 80° C. The resulting mixture was allowed to cool to 5° C. and q.s. with water for losses due to evaporation. The sweeteners and potassium sorbate were then added to the aqueous phase and mixed thoroughly.

B) The film-forming ingredients, xanthan gum, locust bean gum, carrageenan and pullulan were added slowly into a separate container, and rapidly mixed together to form a film forming mixture. The mixture was mixed overnight at a low speed.

C) The film forming mixture was slowly added to the aqueous phase of A), followed by overnight mixing at a low mixing rate to provide a hydrated polymer gel.

D) In another container, the alcohol was mixed with menthol. Physcool was then added to the resulting mixture and mixed. Mono ammonium glycyrrhizinate, polysorbate 80, Atmos 300 and flavors were added to the mixture and further mixed to yield uniformity. Glycerine and mannitol were added to the mixture and mixed.

E) The mixture of step D) was added to the hydrated polymer gel of step C) and mixed uniformly to yield a final polymer gel mixture. The final polymer gel mixture was poured on a mold and cast to form a film of a desired thickness at room temperature. The film was dried under warm air and cut to a desired dimension (dictated by e.g., dosage and mouthfeel).

TABLE 16

| | | % w/w* | % w/w | |
|---|---|---|---|---|
| Material | mg/dose* | Dry Film | Actual Batch | g/batch |
| Dextromethorphan | 15.0000 | 21.4286 | 9.2666 | 11.4615 |
| HBr | | | | |
| Amberlite IRP69 | 16.0000 | 22.8571 | 9.8843 | 12.2256 |
| Xanthan Gum | 0.0944 | 0.1348 | 0.0485 | 0.0600 |
| Locust Bean Gum | 0.1101 | 0.1573 | 0.0566 | 0.070 |
| Carrageenan | 0.4718 | 0.6740 | 0.2425 | 0.3000 |
| Pullulan | 25.1613 | 35.9447 | 12.9359 | 16.0000 |
| Potassium Sorbate | 0.0944 | 0.1348 | 0.0485 | 0.0600 |
| Acesulfame Potassium | 0.7863 | 1.1233 | 0.4042 | 0.5000 |
| Salt | | | | |
| Aspartame NF | 2.2016 | 3.1452 | 1.1319 | 1.4000 |
| Purified water | — | — | 56.7561 | 70.2000 |
| Alcohol USP | — | — | 4.0425 | 5.000 |
| Physcool | 0.1573 | 0.2247 | 0.0808 | 0.1000 |
| Menthol | 2.3589 | 3.3698 | 1.2127 | 1.5000 |
| Peppermint Flavor | 0.1573 | 0.2247 | 0.0808 | 0.1000 |
| Raspberry Flavor | 0.7863 | 1.1233 | 0.4042 | 0.5000 |
| (Givudan) | | | | |
| Mono ammonium | 0.0157 | 0.0225 | 0.0081 | 0.0100 |
| glycyrrhizinate (MAG) | | | | |
| Polysorbate 80 NF | 0.5504 | 0.7863 | 0.2830 | 0.3500 |
| Atmos 300 | 0.5504 | 0.7863 | 0.2830 | 0.3500 |
| Glycerine | 2.3589 | 3.3698 | 1.2127 | 1.5000 |
| Mannitol USP | 3.1452 | 4.4931 | 1.6170 | 2.0000 |
| Total | 70.0000 | 100.0000 | 100.0000 | 123.6872 |

*Assuming that all water is evaporated

The forgoing discussion discloses and describes merely exemplary embodiments of the present invention. One skilled in the art will readily recognize from such discussion, and from the accompanying claims, that various changes, modifications, and variations can be made therein without departing from the spirit and scope of the invention as defined in the following claims.

We claim:

1. A consumable film adapted to adhere to and dissolve in the oral cavity of a warm-blooded animal including humans, comprising at least one water soluble polymer, a taste masking effective amount of a sucralose, and a pharmaceutically active agent.

2. The consumable film of claim 1, further comprising a second sweetener selected from the group consisting of saccharin, aspartame, xylitol, acesulfame potassium and mixtures thereof.

3. The consumable film of claim 1 wherein the sweetener is present in the amount of from about 0.1% to 10% by weight based on the total weight of the consumable film.

5. The consumable film of claim 1 wherein the sweetener is present in the amount of from about 2% to 4% by weight based on the total weight of the consumable film.

6. The consumable film of claim 1 wherein the pharmaceutically active agent is selected from the group consisting of antimicrobial agents, non-steroidal anti-inflammatory agents, antitussives, decongestants, anti-histamines, expectorants, anti-diarrheals, histamine II receptor antagonists, proton pump inhibitors, central nervous system agents, analgesics, antiparkinsonism drugs, narcotic analgesics, analgesics-antipyretics, psychopharmacological drugs and mixtures thereof.

7. The consumable film of claim 1 wherein the pharmaceutically active agent is selected from the group consisting of triclosan, cetyl pyridium chloride, domiphen bromide, quaternary ammonium salts, zinc compounds, sanguinarine, fluorides, alexidine, octonidine, EDTA and mixtures thereof.

8. The consumable film of claim 1 wherein the pharmaceutically active agent is selected from the group consisting of aspirin, acetaminophen, ibuprofen, ketoprofen, diflunisal, fenoprofen calcium, naproxen, tolmetin sodium, indomethacin, flurbiprofen sodium, celecoxib, rofecoxib and mixtures thereof.

9. The consumable film of claim 1 wherein the pharmaceutically active agent is selected from the group consisting of benzonatate, caramiphen edisylate, menthol, dextromethorphan hydrobromide, chlophedianol hydrochloride and mixtures thereof.

10. The consumable film of claim 1 wherein the pharmaceutically active agent is selected from the group consisting of pseudoephedrine hydrochloride, phenylepherine, phenylpropanolamine, pseudoephedrine sulfate and mixtures thereof.

11. The consumable film of claim 1 wherein the pharmaceutically active agent is selected from the group consisting of brompheniramine maleate, chlorpheniramine maleate, carbinoxamine maleate, clemastine fumarate, dexchlorpheniramine maleate, diphenylhydramine hydrochloride, azatadine maleate, diphenhydramine citrate, diphenylpyraline hydrochloride, doxylamine succinate, promethazine hydrochloride, pyrilamine maleate, tripelennamine citrate, triprolidine hydrochloride, acrivastine, loratadine, brompheniramine, dexbropheniramine, fexofenadine, cetirizine and mixtures thereof.

12. The consumable film of claim 1 wherein the pharmaceutically active agent is loperamide.

13. The consumable film of claim 1 wherein the pharmaceutically active agent is selected from the group consisting of famotidine, ranitidine and mixtures thereof.

14. The consumable film of claim 1 wherein the pharmaceutically active agent is selected from the group consisting of omeprazole, lansoprazole and mixtures thereof.

15. The consumable film of claim 1 wherein the at least one water soluble polymer is selected from the group consisting of pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymers, carboxyvinyl polymers, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof.

16. The consumable film of claim 1 wherein said at least one water soluble polymer is pullulan.

17. The consumable film of claim 1 wherein the pharmaceutically active agent is at least one essential oil.

18. The consumable film of claim 17 wherein the at least one essential oil is selected from the group consisting of thymol, menthol, methyl salicylate (wintergreen oil), eucalyptol, carvacrol, camphor, anethole, carvone, eugenol, isoeugenol, limonene, osimen, n-decyl alcohol, citronel, a-salpineol, methyl acetate, citronellyl acetate, methyl eugenol, cineol, linalool, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, clove oil, coal tar, eucalyptus oil, guaiacol, lavender oil, mustard oil, phenol, phenyl salicylate, pine oil, pine needle oil, sassafras oil, spike lavender oil, storax, thyme oil, tolu balsam, terpentine oil, clove oil, and combinations thereof.

19. The consumable film of claim 17 wherein the at least one essential oil is selected from the group consisting of thymol, methyl salicylate, eucalyptol, menthol and mixtures thereof.

20. The consumable film of claim 17 wherein said at least one essential oil is in an antimicrobial effective amount from about 0.01% to 27% by weight based on the total weight of the consumable film.

21. The consumable film of claim 17 wherein the antimicrobial effective amount of the at least one essential oil is from about 0.05% to 18% by weight based on the total weight of the consumable film.

22. The consumable film of claim 17 wherein menthol is a concentration from about 0.01% to 15% by weight based on the total weight of the consumable film.

23. The consumable film of claim 1 is in the form of a single layer.

24. The consumable film of claim 1 further comprising nutritionally acceptable components selected from the group consisting of vitamins, minerals, trace elements, and fibers, soluble fibers and combinations thereof.

25. A method for delivering and enhancing the retention of a pharmaceutically active agent within the oral cavity of a warm-blooded animal including humans comprising administering the consumable film of claim 1 to the oral cavity.
